(19) United States
(12) Patent Application Publication
(10) Pub. No.: US 2010/0249668 A1
(43) Pub. Date: Sep. 30, 2010

(54) ULTRASONIC TRANSDUCER

(76) Inventors: Xiao Dong Zhou, Shenzhen (CN); Florian Steinmeyer, Herzogenaurach (DE); Cheng Ni, Shenzhen (CN)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/712,444

(22) Filed: Feb. 25, 2010

(30) Foreign Application Priority Data
Feb. 25, 2009 (CN) 200910004441.8

(51) Int. Cl. A61N 7/00

(52) U.S. Cl. 601/2

(57) ABSTRACT

An ultrasonic transducer has at least two sets of transducer units, with all the transducer units in each set having the same focus position and the transducer units in the different sets having different focus positions. Since the focus points of the sets of ultrasonic transducer units are distributed in a relatively large spatial range, the range of ultrasonic therapy can be increased even within the narrow and limited space of magnetic resonance imaging equipment.

### BACKGROUND OF THE INVENTION

1. Field of the Invention

The present invention relates to the field of ultrasonic transducers and, particularly, to a multi-focus ultrasonic transducer.

2. Description of the Prior Art

In treatment of a patient with high-intensity focused ultrasound (HIFU) monitored by magnetic resonance imaging (MRI), magnetic resonance imaging equipment can be used to measure the temperature of the heated area of HIFU equipment and to monitor the anatomical image. The focus of the ultrasonic transducer can be made to cover a certain volume of the therapeutic area by the mechanical three-dimension movement of the ultrasonic transducer (treatment head). Since the field of view (examination volume) of the magnetic resonance imaging equipment is usually relatively narrow, the movement range of the ultrasonic transducer is made to be very limited. For a tumor with a relatively large volume to be treated, it is very difficult to carry out the thermal therapy for the whole volume to be treated only by moving the ultrasonic transducer, which makes the treatment of the tumor with a relatively large volume complicated and difficult.

Several methods may be used to solve the above problem. Among them, one method is to use a phase-controlled array of ultrasonic transducers. Since the position of the focus of the phase-controlled array of ultrasonic transducers can be moved within a certain range, the therapeutic range of the ultrasonic transducer can be increased to a certain extent. However, in order to ensure the appropriate form of the focus of the ultrasonic transducer, the focal length can only be changed within a very limited range. In other words, the method is very limited in increasing the therapeutic range.

Another method is to use the technology of acoustic lenses to produce a number of ultrasonic focus points, so that a slightly large heated area is formed by the multiple ultrasonic focus points. However, in the multiple ultrasonic focus points produced by the acoustic lens, the ultrasound output at each ultrasonic focus point cannot be controlled independently. In practice, in order to carry out therapy to a tumor, and at the same time to avoid the heating of normal tissues, there is a need to control independently the power output at each focus point and to control independently the on/off of each focus point.

Still another method is to reposition the patient to carry out repeated treatments. For example, the positions of the ultrasonic transducer and the tumor are changed by adding cushions, so as to increase the therapeutic range. Since in this method it is necessary to reposition the patient, the time for treatment and the complexity of the treatment are increased.

### SUMMARY OF THE INVENTION

In view of the situation, the present invention provides a multi-focus ultrasonic transducer for increasing the range of ultrasonic therapy.

The present invention provides an ultrasonic transducer, having at least two sets of transducer units, with all transducer units in each set having the same focus position and the transducer units in the different sets having different focus positions.

Preferably, each set of the transducer units has an independent power driver unit.

The distance between any adjacent two focus points furthermost in any direction is less than or equal to the movement range of said ultrasonic transducer in that direction.

The focus points of the sets of transducer units are located on the same straight line.

Furthermore, the focus points of the sets of transducer units are located on the axis of said ultrasonic transducer.

The conjunctions of the neighboring transducer units are arranged continuously. Alternatively, the conjunctions of the neighboring transducer units are arranged in a hopping manner.

In one embodiment, the transducer units are divided along radial directions of the ultrasonic transducer.

Furthermore, in this embodiment, the sets of the transducer units are arranged alternately in a tangential direction on the periphery of the ultrasonic transducer.

In another embodiment, said transducer units are arranged coaxially.

Furthermore, in this embodiment, the sets of ultrasonic transducers are arranged alternately.

In still another embodiment, said transducer units are arranged arbitrarily.

Furthermore, in the present embodiment, said transducer units are of an arbitrary shape.

Because the transducer units of the ultrasonic transducer in the present invention are divided into a number of sets, with the transducer units in each set having the same focus position while the transducer units in different sets have different focus positions, and the focus points are distributed in a relatively large spatial range, the range of ultrasonic therapy can be increased even within the narrow and limited space of the magnetic resonance imaging equipment. A number of sets of transducer units can simultaneously output power to carry out treatment at different locations, so that the treatment efficiency is increased and the treatment time is reduced. Since the ultrasonic transducer in the present invention can heat a relatively large space range, the therapeutic schemes for a large tumor can be simplified, and the overall treatment of the tumor can be completed by positioning the patient once. The present invention is not limited to use in the HIFU equipment monitored by magnetic resonance; it is also applicable to other HIFU equipment with a narrow mechanical space.

In addition, the ultrasonic transducer in the present invention can not only be applied to a barrel-shaped superconducting magnet, but also to other types of magnet designs, such as a C-shaped permanent magnet design. Also, the present invention is not limited to a circular ultrasonic transducer design; it is applicable to ultrasonic transducer designs of arbitrary shapes.

### BRIEF DESCRIPTION OF THE DRAWINGS

FIG. 1 is a schematic diagram of the arrangement of the transducer units in an ultrasonic transducer according to the present invention, in which, the ultrasonic transducer has two sets of transducer units E1 and E2, with E1 being composed of transducers D11, D21, D31 and D41, and E2 being composed of transducers D12, D22, D32 and D42.

FIG. 2 is another schematic diagram of the arrangement of the transducer units in an ultrasonic transducer according to the present invention, in which, the ultrasonic transducer has two sets of transducer units E1 and E2, with E1 being composed of D11 and D21, and E2 being composed of D12 and D22.

FIG. 3 is still another schematic diagram of the arrangement of the transducer units in an ultrasonic transducer according to the present invention, in which, the ultrasonic transducer has three sets of transducer units E1, E2 and E3, with E1 being composed of D1, D3 and D7, E2 being composed of D2, D5 and D8, and E3 being composed up of D4 and D6.

FIG. 4 is a schematic diagram showing hopping at the conjunctions of the neighboring transducer units.

FIG. 5 and FIG. 6 are about two neighboring transducer units D1 and D2 in the XOY coordinate system, in which, D1 is located at the center in FIG. 5, and neither D1 nor D2 is located at the center in FIG. 6.

FIG. 7 is a schematic diagram showing a plurality of power driver units.

FIG. 8 is a schematic diagram of the heated area of an ultrasonic transducer having two sets of transducer units, in which, 110 represents a magnet cavity, 120 represents the body of a patient, 130 represents the tumor to be heated, 140 represents water, 150 represents the ultrasonic transducer located at the uppermost end position, and 150′ represents the ultrasonic transducer located at the lower-most end position.

### DESCRIPTION OF THE PREFERRED EMBODIMENTS

The geometric shape of a conventional single-focus ultrasonic transducer is a concave spherical surface, with the center of the sphere being the geometric focus of the ultrasonic transducer, and the curvature radius of the sphere being the focus length. The ultrasonic transducer proposed in the present invention is not limited to a concave spherical shape, instead it can be of an arbitrary shape. However, for the easy description of the technical solution of the present invention, a concave spherical shape will be taken mainly as an example in the following description.

In an embodiment of the present invention, an ultrasonic transducer is divided into n (n≧2) sets of transducer units, with all the sets of transducer units being designed with a concave spherical surface, and the transducer units in the same set having the same focus position, and the transducer units in different sets having different focus positions. For convenience, the sets of transducer units are designated as E1, E2, . . . , En, and preferably the focus points of the sets of transducer units are located on the same straight line, for example, all the focus points are located on the axis of the ultrasonic transducer, so that it is convenient for design and use, but it is not necessary for the focus points of the sets to be on the same straight line. Each set of the transducer units has an independent power driver unit, so as to ensure the power of each set of the transducer units to be outputted independently.

Driven by a mechanical device, the ultrasonic transducer can move along the axial direction of the ultrasonic transducer, and assuming that d is a range of the ultrasonic transducer's axial movement, then the therapeutic range of the ultrasonic transducer is d in the axial direction for a single-focus ultrasonic transducer. However, as to the design of the ultrasonic transducer proposed in the present invention, it is necessary that the distance between the focus positions of any two sets of transducer units with adjacent focus points is less than or equal to d. In this way, the continuity of the therapeutic area can be ensured, thereby avoiding any gap which cannot be treated. Therefore, the therapeutic range of the ultrasonic transducer of the present invention is d+ΔL in the axial direction, wherein, d is the axial movement range of the ultrasonic transducer, and ΔL is the spacing between the two focus points which are furthermost from each other in the axial direction.

As examples, for the structures of the transducer unit sets in an ultrasonic transducer, there are several modes explained below.

Mode 1: the Transducer Units in an Ultrasonic Transducer are Divided Radially in the Ultrasonic Transducer.

There are m×n transducer units divided in the radial directions of the ultrasonic transducer (where, m≧1, and n≧2), and for the sake of convenience these transducer units are sequentially designated clockwise as D11, D12, . . . , D1n, D21, D22, . . . , D2n, . . . , Dm1, Dm2, . . . , and Dmn.

These m×n transducer units are grouped into the n sets as described above, for example, the manner of grouping is as follows:

set E1 includes D11, D21, D31, . . . , Dm1;

set E2 includes D12, D22, D32, Dm2;

. . . ; and

set En includes D1n, D2n, D3n, . . . , Dmn.

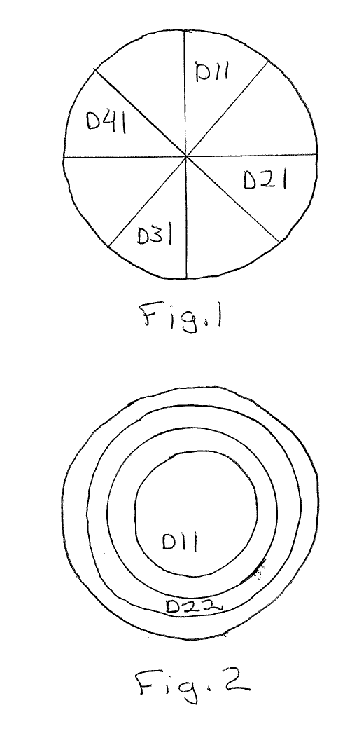

FIG. 1 shows a particular example comprising two sets of transducer units following mode 1. As shown in FIG. 1, the ultrasonic transducer is divided into two sets of transducer units E1 and E2 in radial directions, with E1 including transducer units D11, D21, D31 and D41, and E2 including transducer units D12, D22, D32 and D42. Each set of transducer units has an independent power driver unit, for example, E1 has a power driver unit PD1 (not shown in the figure), and E2 has a power driver unit PD2 (not shown in the figure). As described above, the position of the focus F1 of E1 is different from the position of the focus F2 of E2.

Mode 2: the Transducer Units are Arranged Coaxially.

There are m×n transducer units coaxially divided along the axial direction of the ultrasonic transducer (wherein, m≧1, and n≧2), assuming that these transducer units are sequentially designated as D11, D12, . . . , D1n, D21, D22, . . . , D2n, . . . , Dm1, Dm2, . . . , Dmn from the center outwards.

These m×n transducer units are grouped into n sets as described above, for example, the manner of grouping is as follows:

set E1 includes D11, D21, D31, . . . , Dm1;

set E2 includes D12, D22, D32, Dm2;

. . . ; and

set En includes D1n, D2n, D3n, . . . , Dmn.

FIG. 2 shows a particular example with two sets of transducer units following mode 2. As shown in FIG. 2, the ultrasonic transducer is coaxially divided into two sets of transducer units E1 and E2, with E1 including transducer units D11 and D21, and E2 including transducer units D12 and D22. Each set of transducer units has an independent power driver unit, for example, E1 has a power driver unit PD1 (not shown in the figure), and E2 has a power driver unit PD2 (not shown in the figure). As described above, the position of focus F1 of E1 is different from the position of focus F2 of E2.

Mode 3: the Transducer Units are Arranged Arbitrarily.

The ultrasonic transducer is divided into m transducer units (m≧2) D1, D2, . . . , Dm as required by the design, with the shape of each of the transducer units being designed as an arbitrary one according to actual requirements and design, and then these transducer units are combined into n sets of transducer units as described above.

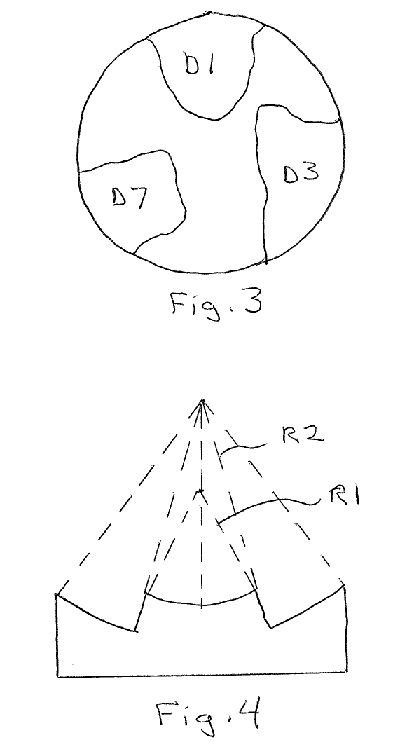

FIG. 3 shows a particular example comprising three sets of transducer units following mode 3. As shown in FIG. 3, the ultrasonic transducer is divided into three sets of transducer units E1, E2 and E3 as required by the design, with E1 including transducer units D1, D3 and D7, E2 including transducer units D2, D5 and D8, and E3 including transducer units D4 and D6. Each set of transducer units has an independent power driver unit, for example, E1 has a power driver unit PD1 (not shown in the figure), E2 has a power driver unit PD2 (not shown in the figure), and E3 has a power driver unit PD3 (not shown in the figure). As described above, the position of focus F1 of E1, the position of focus F2 of E2, and the position of focus F3 of E3 are different from one another.

Since the positions of the focus points of the sets of transducer units are different, and the curvature radii of the sets of transducer units are required to be different, then it is possible to have the occurrence of hopping at the conjunction of neighboring transducer units.

As shown in FIG. 4, a structure is illustrated that has a hopping at the conjunction of the coaxially divided transducer. In FIG. 4, two sets of transducer units respectively have focus lengths R1 and R2. The transducer unit located at the center is lifted, so as to contribute to the reduction of the whole volume of the ultrasonic transducer and to make full use of the range of focal length.

Furthermore, the present invention has proposed a solution so as to avoid hopping at the conjunctions existing in the design of the coaxially divided ultrasonic transducer.

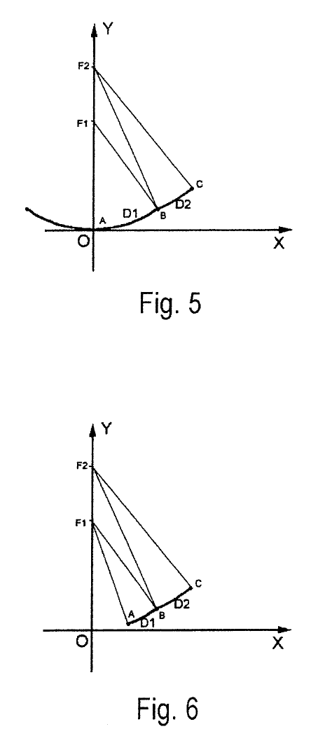

As in the above described m×n sets of transducer units in the coaxially divided solution, as shown in FIG. 5 and FIG. 6, a sectional plane is drawn through the axis of the ultrasonic transducer, and a coordinate system XOY is established, with the intersection point of the axis and the transducer being the coordinate origin O, the radial direction of the transducer being the X axis, the axial direction being the Y axis, and the focus points of all the sets being on the Y axis. Among the m×n transducer units, the distances between any of two neighboring transducer units and the center O are designated as D1, D2, from the near one to the farther one, with their focal positions being (0, F1) and (0, F2), respectively. Their curvature radii can be obtained with the following method:

set the coordinates of the three points A, B, C as (xₐ, yₐ), (x_{b}, y_{b}), (x_{c}, y_{c}), respectively, and if the area D1 is located at the center (as shown in FIG. 5), the curvature radius R₁ of D1 is F1, and the curvature radius R₂ of D2 is R₂=\sqrt{x_{b}²+(F₂−y_{b})²}; and if neither D1 nor D2 is located at the center (as shown in FIG. 6), the curvature radius R₁ of D1 is R₁=\sqrt{xₐ²+(F₁−yₐ)²}, and the curvature radius R₂ of D2 is R₂=\sqrt{x_{b}²+(F₂−y_{b})²}.

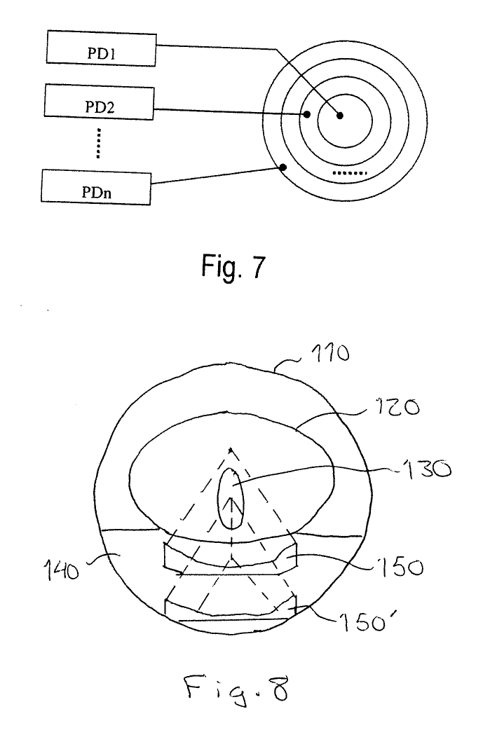

In a preferred case, as shown in FIG. 7, each set of transducer units is supported by one of the independent power driver units PD1, PD2 . . . , PDn, respectively. The output power and the output frequency of each power driver unit are mutually independent, and can be adjusted independently.

As for a relatively large tumor or therapeutic area, the single-focus ultrasonic transducer currently available is not able to cover it. As for the multi-focus ultrasonic transducer provided by the present invention, each set of transducer units has a different focus position, i.e. has a different therapeutic range. Accordingly, during the treatment process, the therapeutic area is first divided according to the therapeutic range of each set of transducer units; then, each respective power driver unit is used to output ultrasonic power to that set or sets of transducer units with the focus points located in the therapeutic area, while that set or sets of transducer units with the focus points outside the therapeutic area is or are turned off.

FIG. 8 shows a schematic diagram of the treatment by two sets of transducer units (with two focus points). Under the condition that the mechanical movement device of the ultrasonic transducer is kept unchanged, a therapeutic range is formed by combining the therapeutic ranges of the short focus length and of the long focus length. If the focus point of only a single set were used, both the therapeutic range of the transducer unit with a long focus length and the therapeutic range of the transducer unit with a short focus length would not be able to cover a relatively large tumor, while the therapeutic range of the combined two sets of focus lengths according to the present invention is capable of covering the whole tumor.

Although modifications and changes may be suggested by those skilled in the art, it is the intention of the inventors to embody within the patent warranted hereon all changes and modifications as reasonably and properly come within the scope of their contribution to the art.

We claim as our invention:

1. An ultrasonic transducer comprising at least two sets of transducer units, the transducer units in each set having the same focus position and the transducer units in the different sets having different focus positions.

2. The ultrasonic transducer as claimed in claim 1, wherein each set of transducer units has an independent power driver unit.

3. The ultrasonic transducer as claimed in claim 1, wherein a distance between the two focus points furthermost in any direction is less than or equal to a movement range of said ultrasonic transducer in that direction.

4. The ultrasonic transducer as claimed in claim 1, wherein the focus points of the sets of transducer units are located on the same straight line.

5. The ultrasonic transducer as claimed in claim 4, wherein the focus points of the sets of transducer units are located on the axis of said ultrasonic transducer.

6. The ultrasonic transducer as claimed in claim 1, wherein the conjunctions of the neighboring transducer units are arranged continuously.

7. The ultrasonic transducer as claimed in claim 1, wherein the conjunctions of neighboring transducer units are arranged in a hopping manner.

8. The ultrasonic transducer as claimed in claim 1 wherein said transducer units are divided along radial directions of the ultrasonic transducer.

9. The ultrasonic transducer as claimed in claim 8, wherein the sets of the transducer units are arranged alternately in a tangential direction on the periphery of the ultrasonic transducer.

10. The ultrasonic transducer as claimed in claim 1 wherein said transducer units are arranged coaxially.

11. The ultrasonic transducer as claimed in claim 10, wherein the sets of ultrasonic transducers are arranged alternately.

12. The ultrasonic transducer as claimed in claim 1 wherein said transducer units are arranged arbitrarily.

13. The ultrasonic transducer as claimed in claim 12, wherein said transducer unit has an arbitrary shape.
